(19) United States
(12) Patent Application Publication
(10) Pub. No.: US 2021/0109012 A1
(43) Pub. Date: Apr. 15, 2021

(54) SAMPLE TESTING APPARATUS

(71) Applicant: G.M.S. Global Mobile Solutions Ltd., Caesarea Industrial Park (IL)

(72) Inventors: Assaf Ben Shoshan, Kibbutz Barkai (IL); Vitaly Strongin, Nesher (IL); Lior Shriki, Haifa (IL); Marcia Deutsch, Caesarea (IL)

(21) Appl. No.: 16/601,684

(22) Filed: Oct. 15, 2019

(51) Int. Cl. G01N 21/17; G01N 33/483; G06T 7/00; H04W 4/80

(52) U.S. Cl. CPC G01N 21/17; G01N 33/483; G06T 7/0012; G01N 2201/0693; G01N 2021/1765; G01N 2201/062; H04W 4/80

(57) ABSTRACT

An apparatus for performing an optical test of a sample comprises a receptacle for accepting a specimen slide, at least one illumination element, an optical lens, and image acquisition apparatus, all having fixed positioned relationships relative to said receptacle.

### FIELD OF THE INVENTION

The present invention relates to apparatus for the inspection of a sample. More particularly, the invention relates to novel apparatus adapted to perform optical analyses of specimens located on a slide. Still more particularly, the invention relates to the use of the novel device for performing a variety of tests, such as fertility tests.

### BACKGROUND OF THE INVENTION

Many different tests require the optical inspection of a specimen, typically place on a transparent slide, to determine a parameter relevant to the specific test undertaken. Illustrative examples of such tests include blood, urine and saliva tests. Another important test is the fertility test to be undertaken by inspecting a variety of sperm parameters. According to the WHO, the following are some of the lowest limits for parameters relevant to the present invention:

Semen volume (ml) 1.5 (1.4 to 1.7)
Total sperm count (10\hat{ }6) 39 (33 to 46)
Sperm concentration (10\hat{ }6/ml) 15 (12 to 16)
Total motility (percent) 40 (38 to 42)
Progressive motility (percent) 32 (31 to 34)
Vitality (percent) 58 (55 to 63)

Other parameters can also be indicative of a fertility problem. Motile sperm concentration (MSC) is an important parameter in assessing whether a fertility problem may exist in a male subject. Detailed MSC analysis can be performed in a laboratory environment and involves expensive and time-consuming procedures. Accordingly, it is desirable to provide a qualitative assessment of MSC, which can be performed at home or at the clinic and can be used for screening purposes. It has been found, according to published statistics and based on the WHO fifth edition for the reference values for semen parameters, that if the sample count is less than 6 M/ml (million per milliliter) this may be indicative of a male fertility problem and requires medical attention. Accordingly, apparatus used for sperm counting must include optical and imaging elements suitable for this level of detection. MSC will be referred to herein as the illustrative test, it being understood that the invention is not limited to any specific test, and that any test requiring optical image acquisition can be performed according to the invention, by providing test-specific parameters such as magnification.

Although devices have been proposed for performing various home tests (the term “home” is used herein to indicate test that can be carried out outside a specialized laboratory, by a nonspecialized individual, such as the subject of the tests, but of course can be performed in any location), for instance by utilizing the camera of a mobile phone, the need still remains for a simple apparatus that delivers consistent results without being influenced by the nature or quality of other devices (such as, for instance, the aforementioned mobile phone.) It is therefore an object of the present invention to provide apparatus that overcomes the disadvantages of prior art devices.

It is a further object of the invention to provide apparatus for performing a variety of tests, without the need of employing highly specialized, complex to use and expensive apparatus.

It is another object of the invention to provide apparatus for the qualitative assessment of fertility of semen, which is simple to operate and which is not influenced by environmental conditions.

Other objects and advantages of the invention will become apparent as the description proceeds.

### SUMMARY OF THE INVENTION

The present invention is directed to apparatus for performing optical testing of a sample. In the description to follow the above will be exemplified by referring to a qualitative assessment of motile sperm concentration of semen, although everything described with respect to this illustrative example applies mutatis mutandis also to other tests. The device according to the invention is characterized by comprising a receptacle for accepting a specimen slide, at least one illumination element, an optical lens, and image acquisition apparatus, all having fixed positioned relationships relative to said receptacle. The fixed position of the elements of the apparatus of the invention are critical to the invention, as will become apparent from the description to follow.

According to one embodiment of the invention, the receptacle is accessed via a slot provided in the outer wall surrounding the apparatus. In a further embodiment of the invention, positioning elements are provided within the receptacle, which are adapted to position the specimen slide in a desired position relative to the optical lens. The positioning elements may comprise both bottom and top positioning elements. In one embodiment of the invention the optical lens is provided in said top positioning element. The at least one illumination element is located below the specimen slide receptacle, to illuminate the specimen images of which are acquired by the image acquisition apparatus.

The apparatus of the invention needs power to operate, and in one embodiment of the invention it comprises a battery suitable to power it. In another embodiment of the invention a power port is provided, which is adapted to supply power to the apparatus from an external source. In a further embodiment of the invention the power port is a USB port.

According to one embodiment of the invention the apparatus comprises circuitry suitable to generate data from the acquired images. In one embodiment of the invention, image processing or other data processing processes are not performed within the apparatus and digital data and/or raw images are transferred to external data processing apparatus for analysis. The external data processing apparatus can comprise any suitable circuitry, and in some embodiments of the invention it is selected from a desktop computer, a laptop computer, a smart phone, a tablet, or the like. However, in some embodiments, image processing circuitry can be provided, to perform at least some processing of the acquired images.

Data can be transferred to processing apparatus or to display means wirelessly, for instance via Bluetooth or Wi-Fi, or via physical connection, for instance via a USB connection. When raw images are transferred to an external device, image processing software is provided in said external device, which is suitable to perform the required analysis of said image, for instance, a qualitative assessment of MSC of semen.

According to another embodiment of the invention the sample assessment (e.g., the qualitative assessment of MSC of semen) is performed in the apparatus, and the apparatus further comprises reporting elements adapted to display a result of the analysis to the user. Said reporting elements may for instance include a display, or one or more lights (e.g., green light for a successful result—i.e. sufficient count—and a red light for an unsuccessful one). In another embodiment of the invention the reporting element is the display or other output means of an external device.

### BRIEF DESCRIPTION OF THE DRAWINGS

In the drawings:

FIG. 1 is a perspective view of apparatus according to one embodiment of the invention, showing a slot adapted to receive a specimen slide;

FIG. 2 is a side view of the device of FIG. 1, further showing the slot opening into a receptacle for a specimen slide;

FIG. 3 (a and b) are side views of the device of FIG. 1;

FIG. 4 is a perspective view of apparatus according to another embodiment of the invention, showing a cable connection port, such as for a USB connection;

FIG. 5 is an exploded view of the apparatus of FIG. 4;

FIG. 6 shows the chassis of the apparatus of FIG. 1 with the top removed and the camera assembly mounted in position;

FIG. 7 is a cross-section of the chassis seen in FIG. 6, taken along the A-A plane;

FIG. 8 is a slightly more elevated view of the cross-section of FIG. 7;

FIG. 9 is a bottom perspective view of the cross-section of FIG. 7; and

FIG. 10 (a and b) show the chassis 101 of the apparatus of FIG. 1, with the LED PCB removed and assembled, respectively.

### DETAILED DESCRIPTION OF THE INVENTION

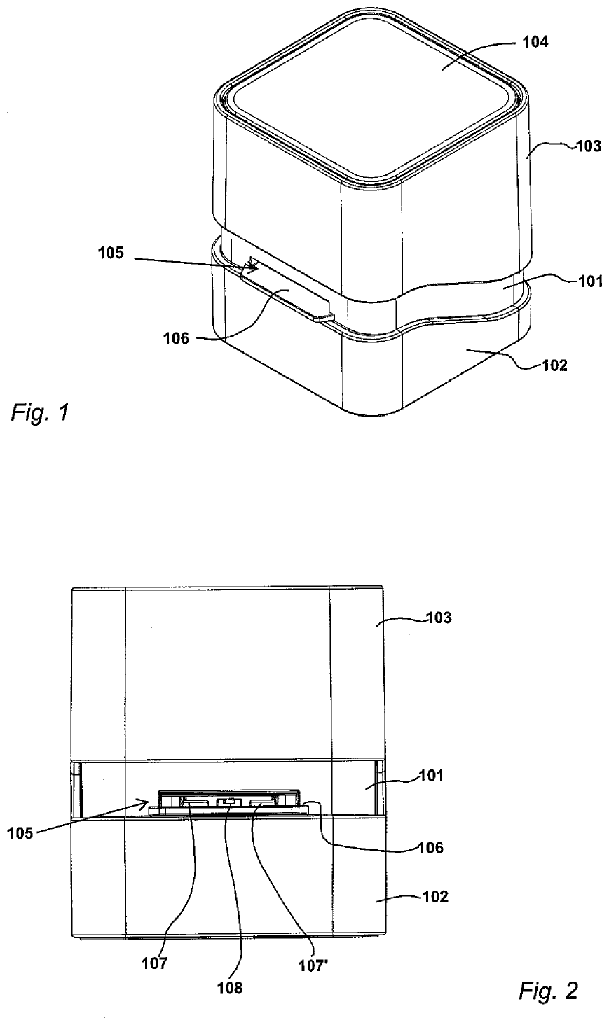

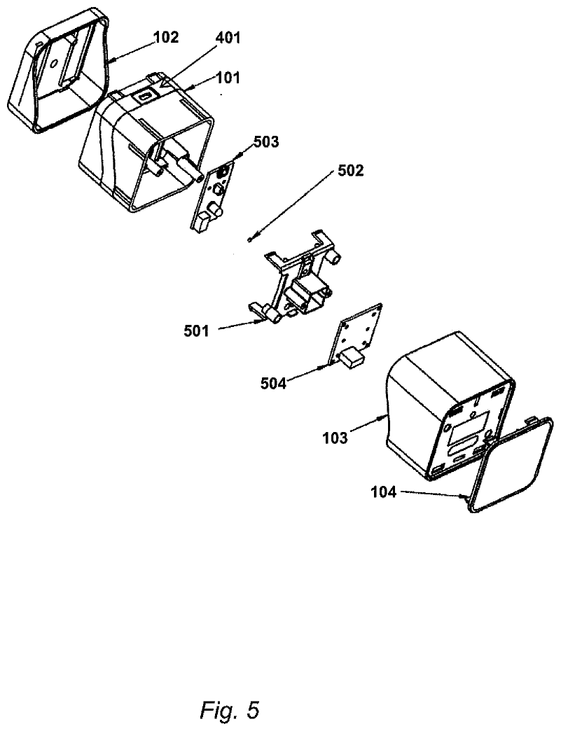

FIG. 1 shows apparatus 100 according to one embodiment of the invention, the internal elements of which will be described in detail with reference to FIG. 5 and following figures. The operating elements are contained in a casing consisting of chassis 101, bottom 102, top 103 and cover 104. A slot 105 is provided, through which a specimen slide can be inserted into the apparatus, and in this particular embodiment a supporting ledge 106 is also seen. Slot 105 is further shown in the side view of the device in FIG. 2, from which internal elements can also be seen. As will be apparent from the description to follow, the appropriate positioning of the specimen slide is critical to the accurate performance of the apparatus. Accordingly, this embodiment of the invention provides for positioning elements 107 and 107′, and 108, which will be further described with reference to FIG. 7.

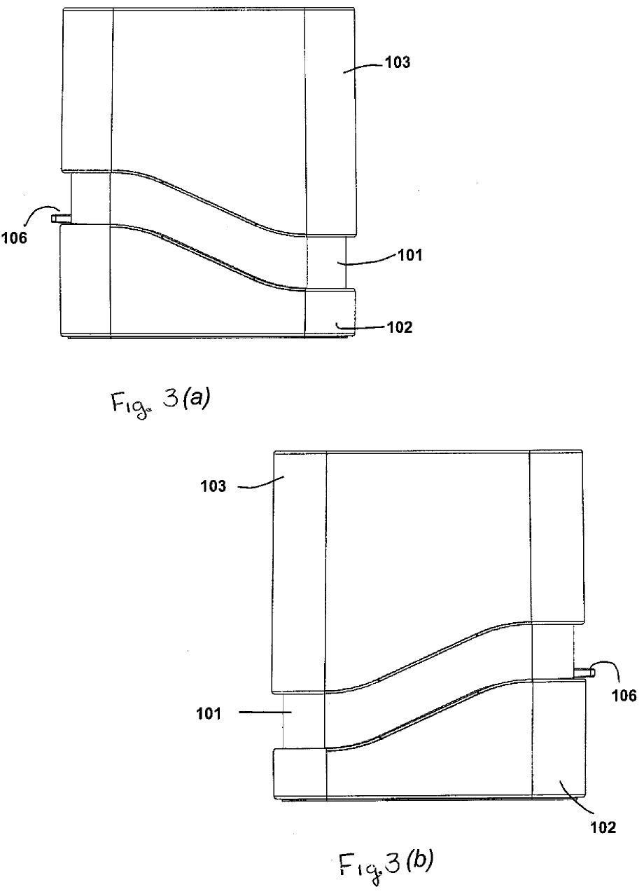

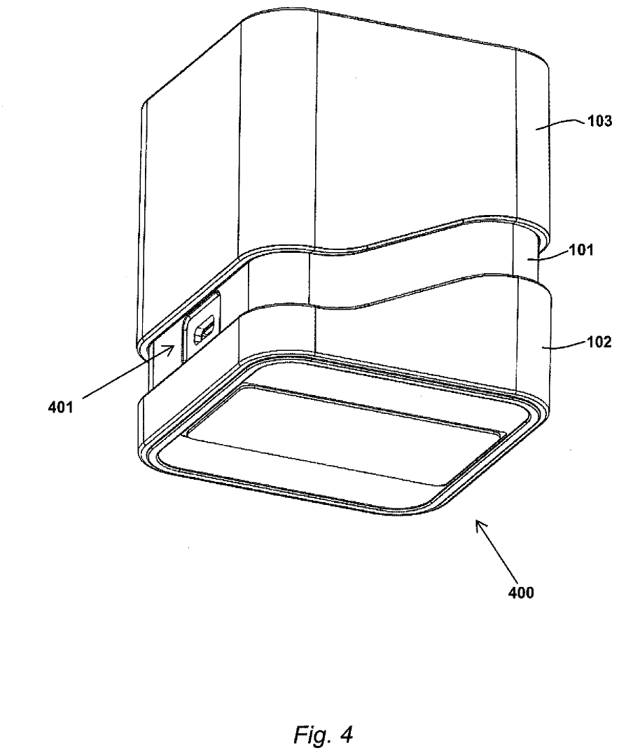

FIGS. 3 (a) and (b) are two side views of the device of FIG. 1, and require no additional explanation.

The apparatus of the invention generates data representative of the sample tested, e.g., MSC. The data so obtained—or the results derived therefrom—needs to be displayed to the user. Said data can be transferred to an external device, such as a PC, smart phone, tablet or the like, via wireless link, e.g. Bluetooth or Wi-Fi, but additionally or alternatively wired connection can also be provided. This is shown in the embodiment of FIG. 4, in which a connection port 401 is provided in device 400. The external elements of device 400 are identical to those of the embodiment of FIG. 1, and therefore the same reference numbers are used to indicate them. Except for the data connection means, the embodiments of FIG. 1 and FIG. 4 are identical and, therefore, they will be referred to alternatively throughout this description.

FIG. 5 is an exploded view of the device of FIG. 4. Lens housing 501 is adapted to house lens 502, which is positioned at a fixed distance from the specimen slide, once it is inserted into the apparatus via slot 105 (FIG. 1). The specimen has to be illuminated, and this is effected by LED PCB 503. As will be apparent to the skilled person, one of the many advantages of the invention is that by providing illumination by LED PCB 503, constant and controlled lighting of the specimen is obtained, without variations that may result from using outside illumination sources, the quality and intensity of which may vary in an uncontrollable manner.

In order to acquire images to be analyzed, a camera assembly 504 is provided, which according to the invention is again positioned at a fixed distance from the specimen slide, thus obtaining a system with constant focus and consistent performance.

The apparatus may be powered by a battery (not shown), or in some embodiments power can be supplied via USB port 401 or through a dedicate power inlet (not shown). Cover 104 is provided on this embodiment to allow access to the top portion of the device, but may not exist in other embodiments.

Any suitable specimen slide can be employed for the purpose of the invention, which will be easily recognized by skilled persons and, therefore, are not discussed herein in detail, for the sake of brevity. An illustrative example of a suitable slide is that sold by MES Global, Los Angeles, Calif., under the name Vision Fixed Coverslip Slides (Item Number: A-CA-01082-00), but any other suitable slide can be used, and the most appropriate slide will be selected by the skilled person for the specific test intended.

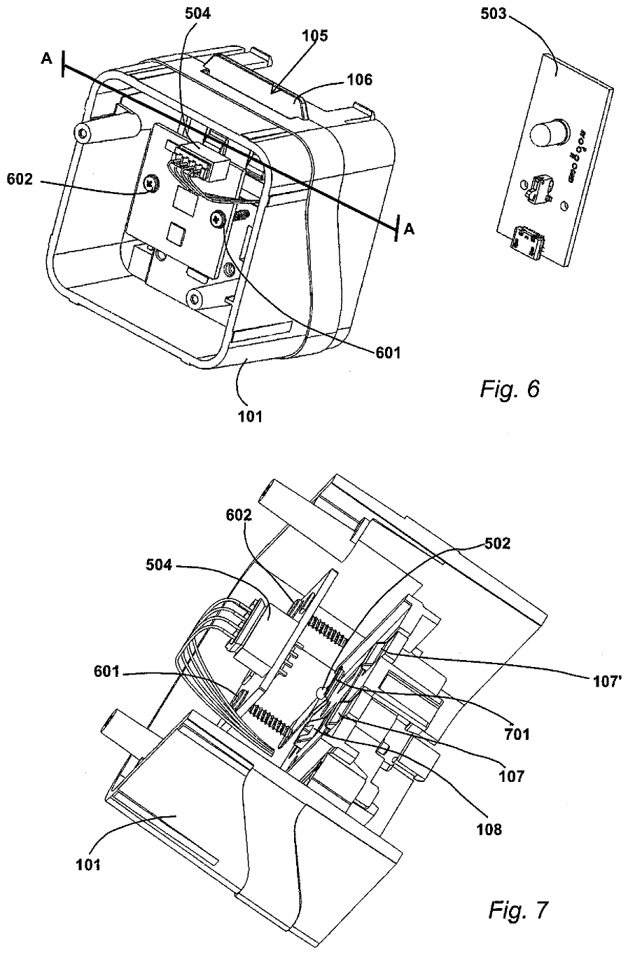

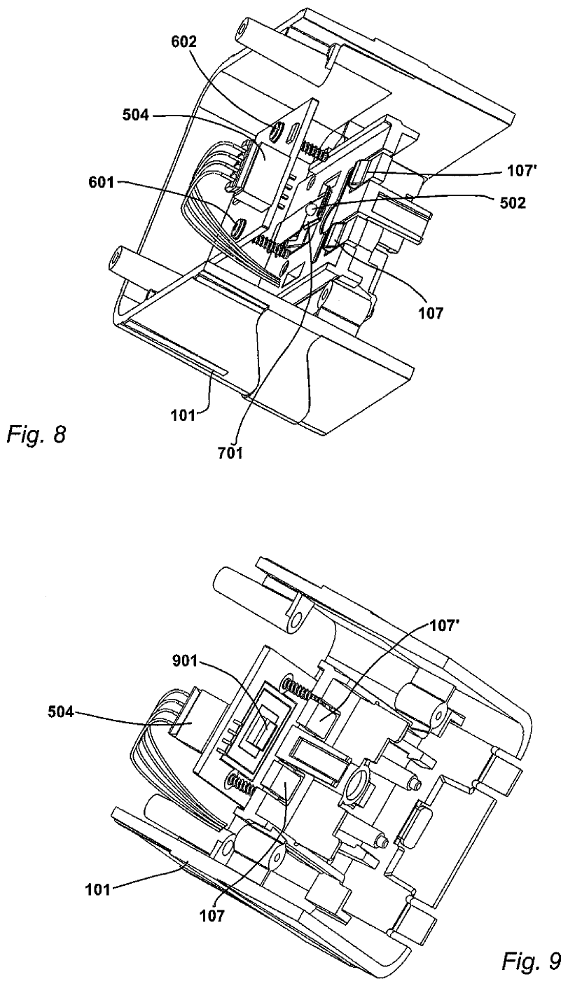

FIG. 6 shows the chassis 101 with the top 103 removed, and the camera assembly 504 mounted in position. The LED PCB 503 is shown as removed from its position below the plane of the test specimen slide. In this exemplary embodiment the camera assembly is held in a fixed place by screws 601 and 602, but of course any other suitable fixation method can be employed. FIG. 7 is a cross-section of the chassis 101 seen in FIG. 6, taken along the A-A plane. In this figure, bottom positioning elements 107, 107′, and 108 are clearly seen, along with top positioning element 701, which also houses lens 502. These positioning elements cooperate in maintaining the test specimen slide in the correct, fixed position for untouched and immediate focus illumination and image acquisition, in a consistent manner across apparatus made according to the invention. As will be appreciated by the skilled person, this fixed positioning is a major advantage of the invention. Said positioning elements can be made to be resilient, if desired, to facilitate the insertion of the test slide. FIG. 8 is a slightly more elevated view of the cross-section of FIG. 7, allowing the same to be viewed in different detail, and FIG. 9 is a bottom perspective view of the cross-section of FIG. 7, in which camera 901 of camera assembly 504 is seen, illustrating its positioned relationship with respect to slot 105 and bottom positioning elements 107 and 107′.

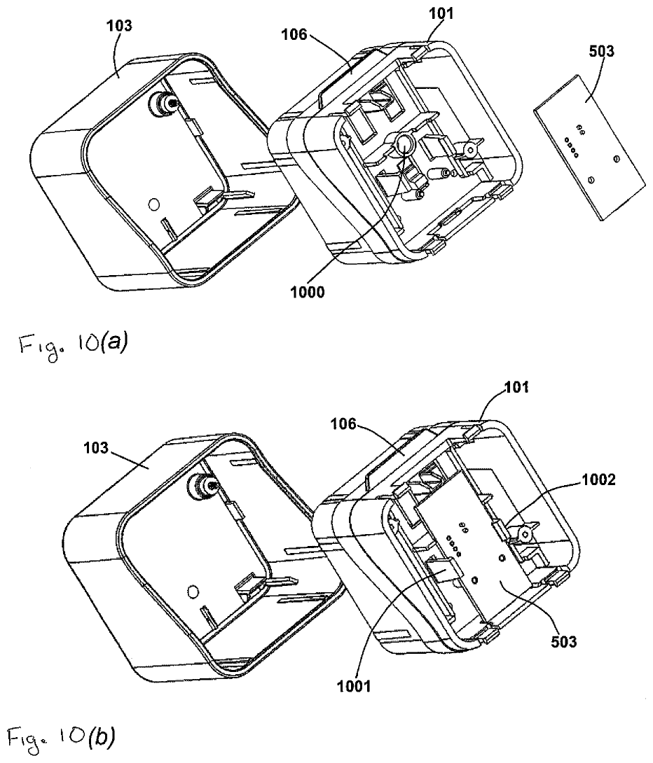

FIG. 10 (a and b) show the chassis 101 with LED PCB 503 removed and assembled, respectively. Illumination is performed through opening 1000 (FIG. 10 (a)) and the PCB is held in place using Snap-on elements 1001 and 1002 (FIG. 10 (b)).

The casing and chassis can be made of any material, and according to one embodiment of the invention they are molded from plastic material such as ABS. Lens 502 can of course be made of any suitable optical material, but according to one embodiment of the invention it is a ball lens with a ×100 optical magnification, images acquired by which can of course be further digitally processed. An illustrative suitable material for this lens is BK7 polished glass. The software used to operate the various components of the apparatus can be easily devised by the skilled person and, therefore, it is not described herein in detail for the sake of brevity. Similarly, the procedure for preparing the test sample slide and for performing the analysis are within the scope of the man of the art and can be, for instance as described in US Patent Publication 2016/0290916 of the same applicant hereof.

All the above description of preferred embodiments has been provided for the purpose of illustration and is not intended to limit the invention in any way, except as provided for in the claims. Many different alternative arrangements to those shown in the figures can be provided. For instance, the chassis and casing may have different shape, the slide receptacle can be differently positioned and, as a result, the positioning of the lens, camera and LED may be different, and many other modifications can be made without exceeding the scope of the invention.

1. Optical testing apparatus, comprising a receptacle for accepting a specimen slide, at least one illumination element, an optical lens, and image acquisition apparatus, all having fixed positioned relationships relative to said receptacle.

2. Apparatus according to claim 1, wherein the receptacle is accessed via a slot provided in the outer wall surrounding the apparatus.

3. Apparatus according to claim 2, wherein positioning elements are provided within the receptacle, which are adapted to position the specimen slide in a desired position relative to the optical lens.

4. Apparatus according to claim 3, wherein the positioning elements comprise bottom and top positioning elements, and wherein the optical lens is provided in said top positioning element.

5. Apparatus according to claim 1, wherein the at least one illumination element is located below the specimen slide receptacle.

6. Apparatus according to claim 1, comprising a battery suitable to power it.

7. Apparatus according to claim 1, comprising a power port adapted to supply power to the apparatus from an external source.

8. Apparatus according to claim 7, wherein the power port is a USB port.

9. Apparatus according to claim 1, comprising circuitry suitable to generate data from the acquired images.

10. Apparatus according to claim 9, wherein data and/or raw images is transferred to external data processing apparatus for analysis.

11. Apparatus according to claim 10, wherein the external data processing apparatus is selected from a desktop computer, a laptop computer, a smart phone, a tablet, or the like.

12. Apparatus according to claim 10, wherein the data is transferred wirelessly.

13. Apparatus according to claim 12, wherein the data is transferred via Bluetooth or Wi-Fi.

14. Apparatus according to claim 12, wherein the data is transferred via physical connection.

15. Apparatus according to claim 14, wherein the physical connection is a USB connection.

16. Apparatus according to claim 10, wherein when raw images are transferred to an external device, image-processing software is provided in said external device, which is suitable to perform an assessment of the sample being tested.

17. Apparatus according to claim 9, wherein the assessment of the sample being tested is performed in the apparatus, further comprising reporting elements adapted to display a result of the analysis to the user.

18. Apparatus according to claim 17, wherein the reporting elements are selected from a display, one or more lights, and the display or other output means of an external device.

19. Apparatus according to claim 1, for performing a qualitative assessment of a fertility parameter, selected from the motile sperm concentration of semen, total sperm count, sperm concentration, total motility, progressive motility, and vitality.

20. Apparatus according to claim 1, for testing a sample selected from blood, urine and saliva.
